(19) United States
(12) Patent Application Publication
(10) Pub. No.: US 2018/0160961 A1
(43) Pub. Date: Jun. 14, 2018

(54) NONINVASIVE AMBULATORY MEASUREMENT OF URINE USING BROADBAND ELECTRICAL SPECTROSCOPY

(71) Applicants: Venugopal Gopinathan, Boston, MA (US); Raghavan Subramanian, Bangalore (IN)

(72) Inventors: Venugopal Gopinathan, Boston, MA (US); Raghavan Subramanian, Bangalore (IN)

(73) Assignees: Venugopal Gopinathan, Boston, MA (US); Raghavan Subramanian, Bangalore (IN)

(21) Appl. No.: 15/665,568

(22) Filed: Aug. 1, 2017

Related U.S. Application Data
(60) Provisional application No. 62/369,767, filed on Aug. 2, 2016.

(51) Int. Cl. A61B 5/20; A61B 5/00

(52) U.S. Cl. CPC A61B 5/204; A61B 5/7275; A61B 5/6823

(57) ABSTRACT

The invention provides a urine sensing device and methods therefor to estimate the volume of urine in a bladder in a non-invasive, real time manner. The urine sensing device comprises plurality of drive electrodes and sensors configured to be positioned in contact with or proximal to skin of the abdomen region of a human such that the relative positions of the drive electrodes and the sensors are known. The plurality of drive electrodes are capable of exciting the abdomen region with an electrical current comprising a plurality of frequencies. The plurality of sensors are capable of measuring at least one electrical parameter from each of the plurality of drive electrodes and the plurality of sensors. Based on the measured values of the at least one electrical parameter, the volume of urine in the bladder is estimated, while taking the electrical conductivity from the bladder tissue, skin, and other extraneous factors into account.

### REFERENCE TO RELATED APPLICATIONS

This application claims the benefit of U.S. Provisional Application No. 62/369,767, filed Aug. 2, 2016.

### FIELD OF THE INVENTION

This invention is related to a method and a device to measure the volume of urine in a person's bladder, the fraction of the bladder that is occupied by urine continuously in an ambulatory and non-ambulatory setting and further providing a time history of these measurements. This has a strong impact in the treatment of all forms of incontinence.

### BACKGROUND

Incontinence is the inability to control the passing of urine which affects quality of life significantly. Without being bound to any theory, the cause of incontinence is due to a disturbance of the delicate balance that exists between the pressure exerted by detrusor muscle and back pressure at the urethral orifice due to the closed urethral sphincter in the urinary bladder. In a normal state, when urine fills the bladder, the bladder expands, which in turn triggers the nerves around the bladder and the brain perceives a need to void the bladder. At the appropriate time, brain relaxes the sphincter and urine is voided. However, in an incontinent state, voiding occurs in an uncontrolled manner leading to a number of problems and thus requiring some lifestyle changes. Some exemplary lifestyle changes and problems include but not limited to, needing proximity to rest-rooms, inability to travel far, smell due to inadvertent leaks, social stigma and associated under confidence, and so on. There are several types of incontinence: urge incontinence, stress incontinence, overflow incontinence and functional incontinence. Some physiological effects of incontinence and their manifestations include: a) Nerve endings erroneously report full-bladder, giving rise to a strong urge to void the bladder; b) Weakness of the urethral sphincter causing an inability to hold back urine even when bladder is not full; c) Insufficient voiding leading to frequent urination; d) Damage to the bladder nerves (injury/accident etc.) leading to an inability to sense urge and backing up of urine into the kidneys.

Currently, incontinence is managed in one or more of the following ways:

Surgical: The objective here is to surgically reduce the overactive bladder muscles by injecting Botulinum toxin (commercially available as Botox®) or collagen into the bladder which tones down the muscles that cause the bladder to contract. This is a partial remedy as typically only a portion of the muscles that are injected are toned down but the remaining muscles that are not injected continue to pose a problem.
Catherization: Tube, with an externally controlled valve (for example Surinate® bladder management system) is used in this procedure. The patient can manually (mechanically or electrically) turn on and off a valve that voids the urine from the bladder through the catheter. The inserted catheter generally poses a level of discomfort to the patient. Further, the inserted catheter becomes a source of infections. Additionally, the periodic maintenance and high cost of this device makes catheters less than ideal in everyday usage scenario for a patient.
Non-surgical: This typically done with what is called a “Bladder diary”. The patient has to record details related to bladder evacuations and accidents such as time of incident. The objective is to gradually stretch the time between evacuations. This is a typical bio-feedback mechanism where the patient is trained to guess incomplete voiding of urine, typical times before which an accident can occur and ultimately develop confidence that his/her brain can indeed control the voiding of urine. This method depends on past history but does not take into account any variations in habits such as excessive or reduced intake of fluids on a given day, which may lead to variation in the volume of the bladder at the predetermined time according to the “Bladder Diary”. The patient receives no real-time actionable information of the state of the bladder as to the level of urine or the degree to which he/she has voided the urine.

Over 30% of men and over 55% of women over the age of 55 experience this problem to varying degrees. The direct costs for the treatment and care of people suffering from incontinence in US alone in 2000 was about $19 billion. When indirect costs are added, the total expenditure increases to over $60 billion. It is estimated that treatment of incontinence is second only to heart-disease in money spent. On top of this, a typical incontinent patient uses over 244 diapers (almost 1 a day) per year.

Thus there is a need for a method and device that can estimate the bladder condition as being full, partially full or empty on a real time basis, which can then be used for further decisions and actions.

### BRIEF DESCRIPTION

In one aspect, the invention provides a urine sensing device configured to be positioned in electrical contact with or proximal to the skin of the abdomen region of a human. The urine sensing device comprises a plurality of drive electrodes capable of exciting the abdomen region with an electrical current comprising a plurality of frequencies. The urine sensing device also includes a plurality of sensors capable of measuring input voltages from each of the plurality of drive electrodes and output voltages from each of the plurality of sensors. The urine sensing device of the invention is configured such that the position of each of the plurality of drive electrodes and the plurality of sensors is known with respect to each other.

In another aspect, the invention provides a method to estimate a volume of urine in a human bladder. The method comprises providing a urine sensing device that comprises a plurality of drive electrodes and a plurality of sensors. The position of each of the plurality of drive electrodes and the plurality of sensors is known with respect to each other. The method then includes placing the urine sensing device such that it is either in electrical contact with or proximal to the skin on the abdomen region of the human. Then, the method involves exciting the abdomen region with the plurality of drive electrodes with a known value of electrical current. The electrical current comprises a plurality of frequencies. The method further comprises measuring at least one electrical parameter at each of the plurality of sensors and then evaluating the volume of urine in the human bladder based on the measured values of the at least one electrical parameter.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic representation of the urine sensing device of the invention;

FIG. 2 a graphical representation of the frequency dependent (magnitude and phase) complex conductivity of various tissue types and urine in the bladder;

FIG. 3 shows error curves for various non-idealities where the actual plant differs from the model being simulated;

FIG. 4 is an illustration of error curves that have been optimized using the method of the invention; and

FIG. 5 is an illustration of error curves that have been optimized when there is an error of 10% in electrode spacing in the plant when compared to the model using the method of the invention.

### DETAILED DESCRIPTION

As used herein and in the claims, the singular forms “a,” “an,” and “the” include the plural reference unless the context clearly indicates otherwise.

As used herein, “urinary bladder” is a hollow muscular organ that collects and stores urine from the kidneys before disposal by urination. In some instances, the term “bladder” is used instead of urinary bladder. Typically, in most animals like humans, the bladder is a hollow muscular, and distensible organ that is positioned on pelvis, and urine enters the bladder via the ureters and exits via the urethra.

“Electrical parameters” as used herein refers to parameters such as current, voltage, resistance, or parameters derived therefrom such as voltage ratios and the like. Other such parameters will become obvious to one skilled in the art and are included within the scope of the invention.

“Electrical network” as used herein includes various electrical circuit elements such as but not limited to resistors, inductors, capacitors, and the like.

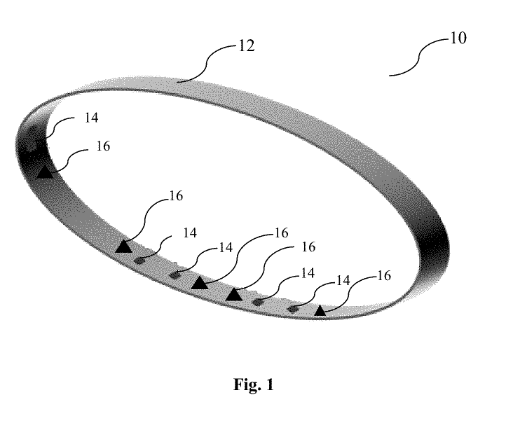

As noted herein, the invention provides a urine sensing device comprising a plurality of drive electrodes. FIG. 1 is a diagrammatic representation of the urine sensing device of the invention, depicted by numeral 10. The urine sensing device is configured to be positioned in contact with or proximal to skin of the abdomen region of a human. The plurality of drive electrodes represented by numeral 14 are capable of exciting the abdomen region with a known electrical current comprising a plurality of frequencies. The urine sensing device also comprises a plurality of sensors depicted by numeral 16. The relative position of each of the drive electrodes and the sensors are known. Each of the sensors is capable of measuring at least one electrical parameter from at least one of the plurality of drive electrodes and each of the plurality of sensors. The urine sensing device may further comprise a known electrical network in the path of the electrical current and a voltage drop sensor to measure the voltage drop across the known resistor as a measure of the current. (not shown in FIG. 1).

Without being bound to any theory, the basis of the functioning of the urine sensing device is that the spectrum of specific conductivity of urine is significantly different from that of all the tissue surrounding the bladder in the abdomen. Thus, any broadband spectrum of voltages measured by any pair of electrical contacts on the skin surface created by exciting the skin surface (not necessarily at the same locations as the points of measurement) will be highly dependent on the amount of urine in the bladder. A single such measurement, however, will give a coarse quantitative measure of relative changes in urine volume and may be prone to gross inaccuracies and variability. By exciting at multiple points and measuring at multiple points again to gain positional diversity and doing this over a wide range of frequencies to gain frequency diversity will increase the accuracy of quantitative measurement. Subsequently, the measured data for every frequency and position can be mathematically fitted to provide a unique solution, which then allows for the estimation of volume of urine. Furthermore with a sufficient number of independent measurements (for the same exact physical condition of the bladder) that these diversities yield, even the electrical parameters that define the specific conductivity of urine and gross tissue surrounding the urine can be estimated accurately, in addition to the volumetric quantities of urine and surrounding tissue.

One skilled in the art will also appreciate that while the urine sensing device of the invention has been described as being in contact with the skin, the device may also be modified to function when in proximity to the skin. The contact may be effected through an extra layer such as a conducting membrane, a conducting gel, or any such layer that does not inhibit the conduction of the electrical pulse through the tissue.

Thus, in another aspect, also as noted herein, the invention provides a method to estimate a volume of urine in a human bladder. The method comprises providing a urine sensing device as described herein. The method involves placing the urine sensing device such that it is either in contact with or proximal to the skin on the abdomen region of the human and exciting the abdomen region with the plurality of drive electrodes with a known value of electrical current comprising a plurality of frequencies. Subsequently, at least one electrical parameter at each of the plurality of sensors is measured which is then used to evaluate the volume of urine in the human bladder. As used herein, electrical parameters also includes the input electric current, which value is used in the estimation of volume of urine in the bladder. As already stated herein, a singular value for the volume of the urine that can satisfy the entire gamut of measurements done over positional and frequency diversity exists, the estimation involves back calculation of the value that fits the measured data. One exemplary technique is impedance tomography. However, mathematically this technique may not be robust enough to yield the right level of accuracy. The method proposed here differs fundamentally from impedance tomography by not solving the “forward problem” (as done in impedance tomography) but by using a simulation feedback loop that actually uses a “reverse computation” to estimate the unknown parameters. This avoids the inherent lack of robustness that is attributed to impedance tomography. This can be achieved in many ways. Some exemplary methods are described herein, however other methods may become obvious to one skilled in the art, are included as part of the invention.

One method involves setting up a finite element analysis engine that solves Maxwell's equations given a gross anatomical modelling of the abdomen, leaving the volume of urine and the volumetric dimensions as variables. For a set of frequency and positionally diverse measurements taken at an instant, a sequence of finite element (FE) modelling runs are conducted, optimizing for changes in the unknowns in the finite element modeling (FEM) simulation (namely the volumes of urine, specific electrical properties of urine and gross tissue and bone masses surrounding the bladder) until the measured set of voltages and current matches to that of the estimated value based on the FEM simulation. When that matching happens to the required degree of precision, the actual volumes used by the FEM simulation algorithm is indeed the actual volume of urine present in the patient. This set of real-time FEM analysis can be performed either in the remote server and the results transmitted back to the display device or they can be performed in the device with the patient.

Another method for estimating the volume of urine in a bladder involves setting up a distributed electrical network of lumped complex infinitesimal complex-valued elements that describes the anatomy. The unknowns can be incorporated inside of each of the individual lumped elements or as a scaling of the network or a combination of them.

Yet another method for the estimation of volume of urine in a bladder involves providing a lookup table. Such a table can be setup using a finite universe of unknowns using an off-line FEM simulation or a lumped-element simulation using canonical parameters. The estimated volume can be obtained by searching in that space using scaled versions of the canonical parameters along with interpolation or extrapolation between points those are not in the lookup table.

The urine sensing device further comprises a processor capable of processing values to estimate the volume of urine in the bladder using one or more of the aforementioned methods. The execution of the one or more of the methods is achieved in the form of an algorithm that may then be programmed onto a chip using an appropriate programming language. The processor may be an integral part of the device or may be remotely present such as on the cloud. When the processor is provided remote to the device, they may be linked to each other through wired means or wirelessly. The urine sensing device may further comprise a memory unit to store raw and processed data.

The method of the invention can be calibrated against non-idealities such as presence of extraneous matter. For example, using a multitude of driving and sensing elements, it is possible to accentuate the effect of conduction through the skin inside the algorithm. This can be used to de-embed the effect of skin impedance which can change significantly over short periods of time for a given patient.

Also, changes in position of the patient (sitting vs standing for example) can alter geometries of organs in the abdomen. Such geometric variations can be accommodated by sensing the position of the patient (either automatically or manually by the patient) and accounting for the correct anatomical arrangement in the calculations within the model used in simulations.

Breath can also be de-embedded out using the fact that there is a coarse periodicity to its effect on the voltages measured and the calculations can be assigned to a particular point in the phase of the breath cycle or actually use that as another point of diversity of measurement (this may involve a further increase in the FEM modelling of the anatomy).

Other non-idealities like existence of digestive products etc can easily be accounted for in the method of the invention, with an acceptable degree of error.

The plurality of drive electrodes may be provided on a band, generally depicted by numeral 12 in FIG. 1. The band may be provided in various ways, such as an elastic band, a belt, a band on an underwear or a diaper or pants, and so on. It will be obvious to one skilled in the art that the plurality of drive electrodes may be provided on the inside part of the wearable article such as underwear, or on the outside or in a pouched layer covered on the inside and outside portions. If the device is provided such that it is proximal to the skin around the bladder as already described herein, the algorithm is configured to account for such variations. The urine sensing device of the invention may further include some means for communicating some preset messages such as, but not limited to, (a) Volume of the urine including the degree of fullness as a percentage; (b) Stratify the degree of fullness into, for example, “safe”, “moderate” and “dangerous.” (c) Show the level of urine remaining in the bladder after voluntary voiding. The manner of communication may be in the form of a display unit configured to display text or in the form of colors etc., a sound unit that has preset tones for various messages, a vibrator unit capable of vibrating for varying lengths of time and number of times to indicate a particular message, and so on.

Thus, the invention provides a device and a method for non-invasive continuous, real-time measurement of urine volume inside the bladder that provides several advantages to an incontinent patient. The device can flag incomplete voiding of urine in real-time thus taking away any guesswork associated with it. It also avoids frequent urination. The device can enable real time bio-feedback, which in turn enables training the brain to ignore false urges and increase the period between urination while simultaneously flagging real urges due to an unexpected reasonably full bladder. The device of the invention is also capable of stratifying risk due to stress incontinence (for example due to coughing or laughing) by noting the level of fullness thereby allowing preventive measures to be taken before an accident. Further, for functional incontinence patients, a real measure of urine is made available by the device for optimum frequency of catheterization. The urine sensing device of the invention may also serve as an effective than “bladder diary”.

### Example

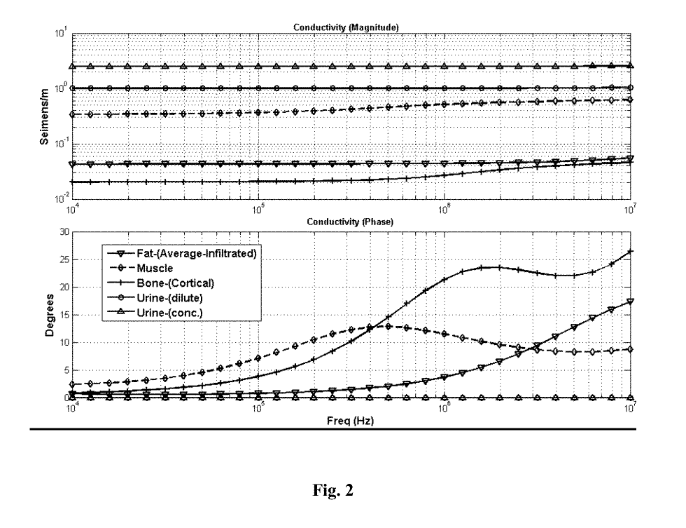

As an illustration of the application of the method of the invention, known values of frequency dependent complex conductivity of various tissue types as described in “Compilation Of The Dielectric Properties Of Body Tissues At RF And Microwave Frequencies” by Camelia Gabriel and Sami Gabriel, Final Report for the Period 15 Dec. 1994-14 Dec. 1995 Prepared for AFOSR/NL Bolling AFB DC 20332-0001 are used herein. FIG. 2 is a graphical representation of the frequency dependent (magnitude and phase) complex conductivity of various tissue types and urine in the bladder.

In the exemplary method to determine the volume of urine in the bladder, a model of the bladder is set up in an electromagnetic (EM) simulator for offline analysis. For the simulation run, bladder is set to contain a volume of 200 mL of urine. A number of parameters of the model, viz. volumetric dimensions and complex conductivities of the bladder, urine and the surrounding tissues that participate in electrical conduction are considered. For a given simulation, the number of parameters N is predetermined. For each of these parameters, the operating range of values are tabulated. Off-line simulations for all combinations of values of each of the N parameters in the operating range are carried out covering all the frequencies of interest to create a large look-up table. Subsequently, a N-dimensional table is generated, which now contains the voltages at each of the electrodes as the simulation output of the model for all possible combinations of parameters and frequencies. For values of parameters that fall in between table entries, an interpolation is performed to obtain the output. Thus the output of the model can be computed using the look up table for a continuous set of parameter values in the operating range. During on-line usage, the objective is to optimize the parameters of the model such that the model output best matches the observed output voltages. To compute the forward pass of the optimizer, instead of running a full EM simulation for a set of parameters, the particular value is picked up from the look-up table after appropriate interpolation. The method involves starting with an initial set of parameters, and modifying them iteratively in order to obtain the best fit between the model output and the observed voltages. Well known optimization methods such as steepest descent, Levenberg-Marquardt, etc may be employed to achieve convergence. Once convergence is achieved, the set of model parameters that correspond to the converged output constitute the solution. Other than the volume of urine, the solution also gives the conductivity of urine, which can also indicate its concentration.

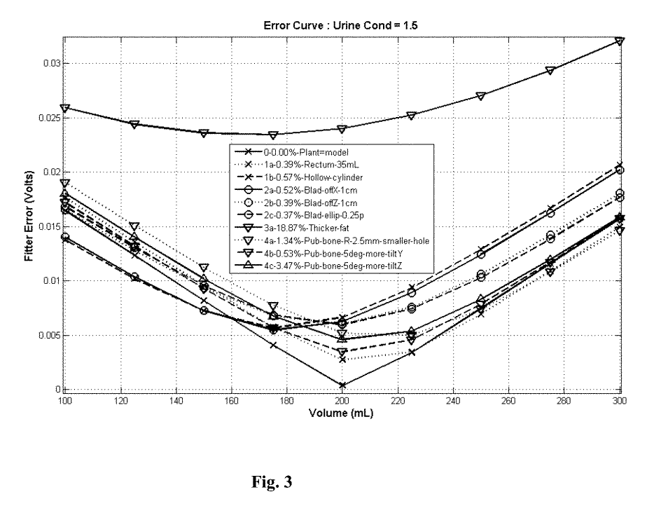

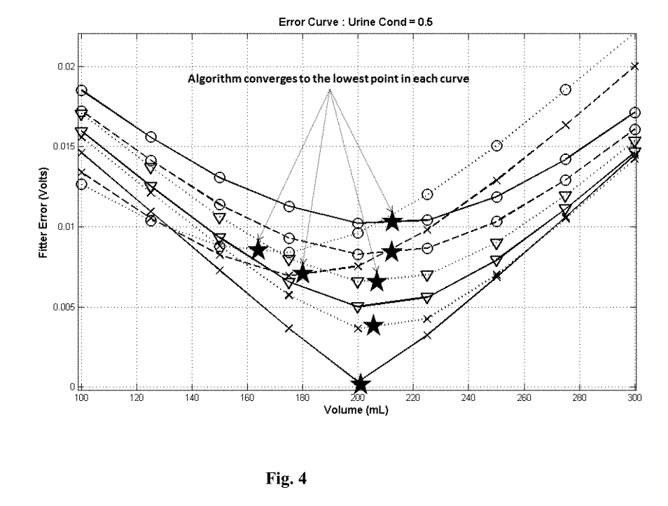

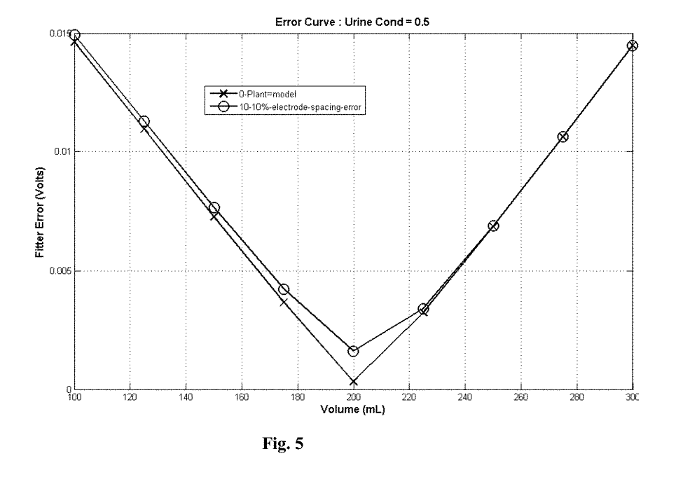

The results of the application of the method as described herein on the model bladder containing a volume of 200 ml of urine are shown graphically in FIG. 3. The plot shows that the application of the method using a computer simulation provides error curves for various non-idealities where the actual plant differs from the model being simulated. FIG. 4 is an illustration of error curves that have been optimized using the method of the invention. Each error curve represents the final converged residual error when all model parameters are optimized except the volume of the bladder (i.e the volume of urine). For purposes of generating the error curve, the urine volume is set to various fixed values and the corresponding residual error is plotted. If the optimization is done by allowing to optimize the volume parameter as well, it would converge to the lowest point on the error curve. It is easy to see that in the presence of many kinds of unmodelled non-idealities, the minima is well within the error bounds of the ideal volume of 200 mL. A similar error curve that has been optimized but with an error of 10% in electrode spacing in the plant when compared to the model is shown in FIG. 5.

Thus, it can be seen that the device and the method of the invention allows for a comprehensive management of the condition of incontinence. It approaches the disease along the proven pathway of bio-feedback to the brain which over time allows the patient to improve the control of the urge to urinate as well as be able to inform the patient when the probability of an accidental voiding is high.

While only certain features of the invention have been illustrated and described herein, many modifications and changes will occur to those skilled in the art. It is, therefore, to be understood that the appended claims are intended to cover all such modifications and changes as fall within the true spirit of the invention.

We claim:

1. A urine sensing device configured to be positioned in contact with or proximal to skin of the abdomen region of a human, wherein the urine sensing device comprises:
a plurality of drive electrodes capable of exciting the abdomen region with an electrical current comprising a plurality of frequencies;
a plurality of sensors capable of measuring input voltages from each of the plurality of drive electrodes and output voltages from each of the plurality of sensors,
wherein the position of each of the plurality of drive electrodes and the plurality of sensors is known.

2. The urine sensing device of claim 1 further comprising a known electrical network in the path of the electrical current and a voltage drop sensor to measure the voltage drop across the known electrical network.

3. The urine sensing device of claim 2 further comprising a processor capable of processing at least one electrical parameter.

4. The urine sensing device of claim 3 wherein the processor is provided on the urine sensing device.

5. The urine sensing device of claim 3 wherein the processor is remote to the urine sensing device.

6. The urine sensing device of claim 5 wherein the processor is connected to the urine sensing device through wired means or wirelessly.

7. The urine sensing device of claim 1 further comprising a memory unit.

8. A method to estimate a volume of urine in a human bladder, the method comprising:
providing a urine sensing device, wherein the urine sensing device comprises a plurality of drive electrodes and a plurality of sensors, wherein the position of each of the plurality of drive electrodes and the plurality of sensors is known;
placing the urine sensing device such that it is either in contact with or proximal to the skin on the abdomen region of the human;
exciting the abdomen region with the plurality of drive electrodes with a known value of electrical current comprising a plurality of frequencies;
measuring at least one electrical parameter at each of the plurality of sensors and the drive electrodes;
estimating the volume of urine in the human bladder based on the values of the at least one electrical parameter.

9. The method of claim 8 wherein the urine sensing device further comprises a known electrical network in the path of the electrical current, and a voltage drop sensor to measure the voltage drop across the known electrical network.

10. The method of claim 9 wherein the estimating is by performing a fine-element analysis to solve Maxwell's equations of electromagnetism by defining the volume of urine in the bladder as an unknown parameter over each of the plurality of frequencies at each position of the drive electrodes and at each position of the sensors based on the at least one electrical parameter.

11. The method of claim 8 wherein the estimating is by performing a lumped circuit element or distributed circuit element approximation of partial differential equations that uses the volume of urine as an unknown parameters over each of the plurality of frequencies at each position of the drive electrodes and at each position of the sensors based on the at least one electrical parameter.

12. The method claim 9 wherein the estimating is based on a lookup-table.
